(19) United States
(12) Patent Application Publication
(10) Pub. No.: US 2014/0271499 A1
(43) Pub. Date: Sep. 18, 2014

(54) ARTICLE AND METHOD OF INITIATING BONE REGROWTH AND RESTORATION OF GUM RECESSION WITH LOCALIZED SUBGINGIVAL DELIVERY OF MEDICATIONS

(71) Applicant: Duane C. Keller, St. Louis, MO (US)

(72) Inventor: Duane C. Keller, St. Louis, MO (US)

(21) Appl. No.: 14/213,041

(22) Filed: Mar. 14, 2014

Related U.S. Application Data
(60) Provisional application No. 61/784,871, filed on Mar. 14, 2013.

(51) Int. Cl. A61K 6/00; A61C 19/06; A61Q 11/00

(52) U.S. Cl. CPC A61K 6/007; A61Q 11/00; A61C 19/063
USPC 424/53; 433/215

(57) ABSTRACT

A system and method of initiating alveolar bone regrowth about a tooth of a patient suffering from bone loss due to periodontal disease that includes topically administering to a periodontal pocket about the tooth of the patient a hydrogen peroxide solution and topically administering to the periodontal pocket about the tooth a medicament that can help manage/control osteogenic activity.

### CROSS-REFERENCE TO RELATED APPLICATIONS

This application claims the benefit of U.S. Provisional Application No. 61/784,871, filed on Mar. 14, 2013, the disclosure of which is incorporated herein by reference.

### FIELD

The present disclosure relates to treatment of periodontal disease and, more specifically, to systems and methods for treating and controlling initiating bone regrowth and restoration of gum restoration that results therefrom.

### BACKGROUND

The statements in this section merely provide background information related to the present disclosure and may not constitute prior art.

It has been found that these oral periopathogens associated with periodontal disease causes bone loss and tissue recession. Mastication on infected gum tissues increases the destruction of bone associated with the diseased teeth. Simple oral hygiene procedures, such as brushing and flossing of the teeth can help with slowing periodontal disease, but does not result in the regrowth of the lost bone. Current methods to treat periodontal disease and the resulting effects suffer from a number of significant drawbacks and are often ineffective in addressing the results from periodontal disease such as bone loss.

Furthermore, bone loss around the teeth has always been considered inevitable. In many cultures it is understood that there is an association between age and the length of a person tooth. In the English tradition, long of tooth has always been associated with advancing age as it was just expected that as you age you will lose more bone support than is replaced and your teeth will appear longer. Normal expectation have been for humans as they age to experience an increased osteoclastic activity as compared with osteoblastic activity and this would result in more bone loss than bone regeneration.

The inventor of the present methods and systems has identified a significant need and desired benefit to many patients in developing new procedures and systems that not only stop periodontal disease but also aid in reversing the effects of the disease by restoring the gums that have receded and also initiating the regrowth of bone.

### SUMMARY

The inventor hereof has succeeded at designing systems and method that are capable of decreasing bone loss and increasing bone regeneration that facilitates the development of new bone. These include reducing/suspending/preventing bone deterioration and inducing/promoting bone regrowth. The inventive methods generally include administering under the course of a therapy an effective amount of directly applied hydrogen peroxide and can also include the direct application of a medicament such as an antibiotic, that in one example is a broad spectrum antibiotic and in some embodiments is a synthetic analog of an antibiotic such as tetracycline and in one embodiment is doxycycline.

The inventive method can be used for treating or preventing conditions associated with periodontal disease and/or aging, such as alveolar bone loss, alveolar bone degradation, and the resulting loss of gingival tissue. This is accomplished through the use of the system and method that can inhibit the function of existing osteoclasts and abrogate the formation of new osteoclasts. Additionally, the present methods and systems have been shown through testing to also promote and/or regenerate the alveolar bone through maintaining and/or increasing the cellular activity of osteoblasts that regenerate bone growth. Such reduction in osteoclasts activity and increase in osteoblasts activity with resulting regenerated bone growth has been found to first decrease bone loss and gingival recession and then to promote and lead to regenerated bone with an increase in gingival tissue and gum tissue on and or otherwise covering the regenerated alveolar bone and about the teeth. The regrowth of the alveolar bone and the gingival tissue further aids in the treatment for periodontal disease. The system and method not only produces improvements in support for the teeth, but esthetically appearing teeth through the regained lost gum tissue, the teeth appear have an appearance of being shorter and also have an improved environment for fighting and maintaining a gum/teeth environment that aids in the reduction of periodontal disease.

In one aspect, a method of initiating bone regrowth in a patient suffering from bone loss and recessed gums about one or more teeth including preparing a periodontal medicament delivery tray for the patient with an application region being configured for applying one or more medicaments to a different one of the treatment regions, one or which is the identified bone regrowth treatment region, placing hydrogen peroxide for micro-organism control and osteoclastic and osteoblastic controlling medications in the treatment region of the tray which includes the identified bone regrowth treatment region, and applying the tray with the medication(s) to the patient treatment region.

In another aspect, a method of initiating alveolar bone regrowth about a tooth of a patient suffering from bone loss due to periodontal disease includes topically administering to a periodontal pocket about the tooth of the patient an osteoclastic and osteoblastic modifying substance that is topically administering to the periodontal pocket about the tooth as a medicament.

In yet another aspect, a method of initiating bone regrowth in a patient suffering from alveolar bone loss and recessed gums about one or more teeth includes the steps or processes of identifying a treatment region for regrowth of the alveolar bone associated the one or more teeth that includes a periodontal pocket therein and preparing a periodontal medicament delivery tray for the patient. The delivery tray is prepared to have a cavity that defines one or more application regions with each application region about the one or more of the teeth in the identified treatment region. The process next includes placing an osteoclastic and osteoblastic controlling solution in the application region of the tray cavity and applying the medication solution placed in the tray cavity to the periodontal pocket in the treatment region. The process further includes placing a medicament in the application region of the tray cavity and applying the medicament placed in the tray cavity to the periodontal pocket in the treatment region.

Various aspects of the present disclosure will be in part apparent and in part pointed out below. It should be understood that various aspects of the disclosure may be implemented individually or in combination with one another. It should also be understood that the detailed description and drawings, while indicating certain exemplary embodiments, are intended for purposes of illustration only and should not be construed as limiting the scope of the disclosure.

### BRIEF DESCRIPTION OF THE DRAWINGS

FIG. 1A is a cross-sectional view of a healthy tooth and gingival tissue.

FIG. 1B is a cross-sectional view of a tooth and gingival tissue in the presence of periodontal disease resulting in considerable alveolar bone loss and associated gingival tissue loss thereon.

FIGS. 2A and 2B are a cross-sectional close-up photographic image (FIG. 2A) and a graphical representation thereof (FIG. 2B) showing a marginal gingiva and tooth structure including a cementoenamel junction.

FIGS. 3A and 3B are a cross-sectional close-up photographic image (FIG. 3A) and a graphical representation thereof (FIG. 3B) is a close-up side photographic image of a tooth and associated marginal gingiva.

FIGS. 4A and 4B are a cross-sectional close-up photographic image (FIG. 4A) and a graphical representation thereof (FIG. 4B) of another close-up side photographic image of a tooth and associated marginal gingiva.

FIG. 5 is a top plan view of a periodontal medicament delivery tray suitable for use with one or more embodiments of the present disclosure.

FIG. 6 is a side elevation view of a periodontal medicament delivery tray as worn by a patient for use in one or more embodiments of the treatments of the present disclosure.

FIG. 7 is a side elevation view of upper and lower periodontal medicament delivery trays as worn by a patient for simultaneous treatment of both the upper and lower teeth and associated marginal gingiva according to some embodiments.

FIGS. 8A and 8B are close-up photographic image (FIG. 8A) and a graphical representation thereof (FIG. 8B) of an initial radiograph taken in 1976 demonstrating the bone level for a 55 year old patient that is beginning to experience bone loss and tissue recession.

FIGS. 9A and 9B are close-up photographic image (FIG. 9A) and a graphical representation thereof (FIG. 9B) of a 2012 radiograph image taken of the same patient of FIGS. 8A and 8B taken in 2012 showing an increase in alveolar bone and gingival tissue.

FIGS. 10A and 10B are side image radiographs (FIG. 10A) and graphical representation thereof (FIG. 10B) of the same patient in 1976 showing the effects of alveolar bone loss and its effect on the loss of gingival tissue due to periodontal disease.

FIGS. 11A and 11B are side image radiographs (FIG. 11A) and graphical representation thereof (FIG. 11B) of the same patient of FIGS. 11A and 11B also taken in 2012 also showing the positive effects of growth of the alveolar bone and growth of gingival tissue thereon due to presently disclosed process.

FIGS. 12A and 12B are a front photographic image (FIG. 12A) and graphical representation thereof (FIG. 12B) of a patient taken in 2004 showing the effects of alveolar bone loss and its effect on the loss of gingival tissue due to periodontal disease before beginning the herein disclosed treatments.

FIGS. 13A and 13B are a front photographic image (FIG. 13A) and graphical representation thereof (FIG. 13B) of the same patient of FIGS. 12A and 12B taken in 2012 after having received the herein described treatments and showing the positive effects of growth of the alveolar bone and growth of gingival tissue thereon demonstrating the effects of the current method of treatment using hydrogen peroxide and doxycycline as disclosed by way of one embodiment.

FIGS. 14A and 14B are a right side photographic image (FIG. 14A) and graphical representation thereof (FIG. 14B) of the patient corresponding to FIGS. 12A and 12B also taken in 2004.

FIGS. 15A and 15B are a right side photographic image (FIG. 15A) and graphical representation thereof (FIG. 15B) of the patient corresponding to FIGS. 14A and 14B also taken 2012 (the same as FIGS. 13A and 13B) after receiving treatment for 8 years.

FIGS. 16A and 16B are a left side photographic image (FIG. 16A) and graphical representation thereof (FIG. 16B) of the patient corresponding to FIGS. 12A and 12B also taken in 2004.

FIGS. 17A and 17B are a left side photographic image (FIG. 17A) and graphical representation thereof (FIG. 17B) of the patient corresponding to FIGS. 16A and 16B also taken 2012 (the same as FIGS. 13A and 13B) after receiving treatment for 8 years.

FIGS. 18A and 18B are a lateral perspective radiograph image (FIG. 18A) and graphical representation thereof (FIG. 18B) of the patient of FIGS. 12A and 12B also taken in 2004 showing the loss alveolar bone and of gingival tissue due to periodontal disease.

FIGS. 19A and 19B are a lateral perspective radiograph image (FIG. 19A) and graphical representation thereof (FIG. 19B) of the same patient of FIGS. 18A and 18B taken in 2012 showing the regrowth of alveolar bone and gingival tissue following treatment using one embodiment of the method described herein.

As noted above, for each radiograph image, the inventor has provided a graphical representation as a “prime” version to aid in the depiction and description of the radiographic image. It should be understood that throughout the drawings, corresponding reference numerals indicate like or corresponding parts and features.

### DETAILED DESCRIPTION

The following description is merely exemplary in nature and is not intended to limit the present disclosure or the disclosure's applications or uses.

In some embodiments as disclosed herein, direct medication application to periodontal pockets using the method as disclosed herein (also referred to as the Perio Protect Method) uses a custom formed medical device (Perio Tray) that has been shown to be superior to conventional scaling and root planning in combination with brushing and flossing. Patients treated with this disclosed antioxidant therapy using the Perio Protect Method have found a reversal of the disease process, augmentation of bone reformation, decreased tooth mobility and the ability to manage periodontal disease long-term. In one embodiment of this method, it has been proven by the inventor hereof that delivering 1.7% hydrogen peroxide (Perio Gel) and a medicament such as doxycycline (Vibramycin Syrup) 50 mg/5 ml to the gingival sulcus or periodontal pocket for treating periodontal disease better than scaling and root planning and doxycycline stops bone loss and augments bone regeneration. The inventor has also shown that other medicaments can also be used that also augment the growth and regeneration of bone. These can include other antibiotics as well as cardiovascular medicaments that can augment the regrowth and/or regeneration of bone. It should be understood to those of skill in the art, that the following descriptions that refers to doxycycline, Vibramycin or an antibiotic, are only used for examples and should be understood to also include any bone regrowth/regeneration medicament.

Case studies have shown that this method and system can provide a significant reversal of bone loss and gum recession over time using a direct medication delivery to the periodontal pocket using the current method (also referred herein to as the “Perio Protect Method”). The resulting newly formed bone with doxycycline is stronger, less oral bone loss is evident, and tooth mobility has been shown to decrease as doxycycline is incorporated onto the bone/root surface. The use of the current method especially when using doxycycline has been shown to be significant factors for managing recession, augmenting bone regeneration, re-supporting teeth that have lost bone support, controlling peri-implantitis and endodontic/periodontic lesions and augmenting bone graft placement and maintenance.

In some embodiments, the combination of the use of a customized prescription tray delivery of medication with SRP and conventional homecare has been shown to produce greater reductions in bleeding on probing and pocket probing depths than SRP alone. The direct medication delivery using the Perio Protect Method employs a doctor prescribed custom formed medical device (Perio Tray) to conform to the scope and magnitude of the patient's periodontal conditions to deliver doctor selected medications to the periodontal pocket or gingival sulcus or region around a patient's teeth or implant. The medications chosen are the agents that are effective in controlling oral biofilm, stopping bone loss and augmenting bone regeneration.

One exemplary method will now be described. An initial examination determines the conditions of the patient's periodontal disease. Models of the patient along with their periodontal probing is forwarded to a laboratory registered to make the custom formed medical device (Perio Tray), which is capable of delivering about 1.7% hydrogen peroxide gel (Perio Gel) to the diseased tissue. The 1.7% hydrogen peroxide Perio Gel penetrates the oral biofilm and eradicates the biofilm within about 15 minutes as hydrogen peroxide has been shown to kill 99.999% of biofilm bacteria. The depth of the biofilm and the tissue damage that exist determines the frequency and duration of usage. Patients with severe tissue damage (6 mm or greater) use the Perio Tray about 4 times a day. Patients with moderate damage (3 mm to 6 mm) use the Perio Tray about 3 times a day and patients maintain their health using the trays once or twice a day for about 15 minutes.

One embodiment includes use of hydrogen peroxide that is one of a few medications that is biocompatible and readily penetrates an oral biofilm. Hydrogen peroxide can provide oxygen and water as it undergoes an oxidative/reduction reaction. This oxidative/reduction reaction forms OH− and H+ radicals that are responsible for the bactericidal effect of hydrogen peroxide. The Perio Gel poloxamers are specifically designed to manage extra radicals to decrease tissue injury. Doxycycline also helps manage oxidative radicals and decreases tissue injury in chronic wounds.

This method has been shown to increase the oxygen saturation level in the periodontal pocket 5.3 times normal, similar to the conditions that are found in a hyperbaric chamber. The oxygen formed is delivered into the gingival sulcus or periodontal pocket and increases the oxygen concentrations, thus converting the periodontal pocket region from one that was anaerobic to one that would have a much higher concentration of oxygen than normal. Repeated treatments would be used to help maintain this oxygen advantage.

The application as described herein using hydrogen peroxide not only penetrates the oral biofilm and is bactericidal, but oxygen under pressure stimulates new cell growth, initiates new blood vessel formation, and cause fibroblasts to make a stronger collagen. Oxygen delivered directly to the tissues with the Perio Protect Method resulting in an increased oxygen concentration that has been shown to kill the obligate anaerobes in 2 days, bring to an end the facultative anaerobes in 7 days and eradicate 99.98% of the biofilm bacteria in 14 to 17 days as it denatured the protein pellicle on the tooth surface. This elimination of the disease causing bacteria and the healing benefits of oxygen would facilitate health and tissue recovery.

The current methods can use a topically applied broad spectrum antibiotic such as doxycycline directly on the gums and into the periodontal pockets has been shown to augment bone regeneration. An initial study has demonstrated a decrease in osteoclastic activity when exposed to doxycycline and an augmentation of osteoblastic activity, resulting in stopping bone loss and initiating bone regeneration. Doxycycline bonds to internal and external calcium ions causing osteoclasts to detach from the bone, thus decreasing or stopping bone loss. Osteoclastic precursor cells when exposed to doxycycline are completely abrogated from making any new osteoclasts, thus continuing the cessation of bone loss. Osteoblasts were found to move to the loci of the detached osteoclasts and they continue to make new bone. The stoppage of bone loss and the maintenance of bone regeneration results in a net gain of new bone over time as long as the doxycycline is continually delivered to the bone/tissue interface with the currently disclosed method.

In one embodiment, the method herein of initiating alveolar bone regrowth about a tooth of a patient suffering from bone loss due to periodontal disease includes topically administering to a periodontal pocket about the tooth of the patient a hydrogen peroxide solution and topically administering to the periodontal pocket about the tooth a medicament.

In another embodiment, the method of initiating bone regrowth in a patient suffering from alveolar bone loss and recessed gums about one or more teeth includes the steps or processes of identifying a treatment region for regrowth of the alveolar bone associated the one or more teeth that includes a periodontal pocket therein and preparing a periodontal medicament delivery tray for the patient. The delivery tray is prepared to have a cavity that defines one or more application regions with each application region about the one or more of the teeth in the identified treatment region. The process next includes placing a hydrogen peroxide solution in the application region of the tray cavity and applying the hydrogen peroxide solution placed in the tray cavity to the periodontal pocket in the treatment region. The process further includes placing a medicament in the application region of the tray cavity and applying the medicament placed in the tray cavity to the periodontal pocket in the treatment region.

The steps of topically administering the hydrogen peroxide solution and topically administering the medicament can each performed through the use of a periodontal medicament delivery treatment tray having a cavity for receiving the tooth a portion of the gum tissue surrounding the tooth including the periodontal pocket. It is within the current disclosure that the administering steps include placement of the hydrogen peroxide solution and the medicament in the cavity and thereafter placement of the periodontal medicament delivery treatment tray about the tooth and surrounding gum tissue, either simultaneously or sequentially during the treatment. The prior providing for placing of the hydrogen peroxide solution in the cavity of the tray and the topically administering of the hydrogen peroxide to the periodontal pocket that is separate and prior to (in most cases but could also be afterwards) the placement of the medicament in the cavity of the tray and the topically administering of the antibiotic to the periodontal pocket.

As it is often desired to apply the hydrogen peroxide and/or medicament into the periodontal pocket, the tray can be prepared to create a positive pressure within the cavity when the tray is placed about the tooth and surrounding gum tissue. In this manner, the treatment compounds can be forced into greater topical contact with the gingival tissue and deeper into the periodontal pocket.

As addressed above, the medicament can be any suitable, but in some embodiments is an antibiotic such as a broad spectrum antibiotic that by way of example can include doxycycline. Where doxycycline has been used, the inventor hereof has tried a concentration ratio of about 50 mg/5 ml and found such to be suitable and effective. However, the inventor believes that other concentrations could also be used as well as other antibiotics which of course could be of different concentrations. The medicament could also be a cardiovascular medicament. The medicament can be chosen without considerable experimentation based on a testing or identification of a medicament that can be delivered topically in the oral cavity and that result in decreasing osteoclasts that are attached to the alveolar bone, decreasing their production and precursors thereto and that also aid, benefit or promote osteoblasts.

The inventor hereof has used a hydrogen peroxide solution of about 1.7% hydrogen peroxide but other concentration is possible. Such can be identified or selected such that the solution provides oxygen such as through an oxidative/reduction reaction. Such an oxidative/reduction reaction can forms OH− and H+ radicals that are can promote the bactericidal effect of hydrogen peroxide. This application can be for any suitable period of time such as applying the hydrogen peroxide solution into the periodontal pocket is for a period of time of at least about 15 minutes.

This method has been shown to increase the oxygen saturation level in the periodontal pocket 5.3 times normal with the formed oxygen being delivered into the gingival sulcus or periodontal pocket thereby increasing the oxygen concentrations therein and converting the periodontal pocket region from one that was anaerobic to one that would have a much higher concentration of oxygen than normal. Repeated treatments would be used to help maintain this oxygen advantage.

The steps of topically administering the hydrogen peroxide and the medicament over an extended period of time can be monitored to determine that bone loss has been stopped and that the alveolar bone about the tooth has grown. This can include identification of the inhibiting of the attachment of osteoclasts to the alveolar bone about the tooth and the promotion of cellular production of osteoblasts.

Referring now to the Figures, FIG. 1A shows a healthy tooth 10 with surrounding gum 12. The tooth 10 includes a crown 14 having enamel 15, an inner composition of dentine 16 that forms the root of the tooth 10, and cementum 18 that covers the outer surface of the lower portions of the tooth 10 below the enamel 15. A cementoenamel junction (CEJ) 20 is defined as the intersection of the lower end of the enamel 15 and the start of the cementum 18. As shown, the gum 12 includes gingiva 22 that has gingival tissue 24 having a portion referred to as marginal gingiva tissue 26, and attached gingiva tissue 28, the two portions generally being separated by the CEJ 20. The gum 12 forms a gingival sulcus 30 at the intersection of the gum 12 with the tooth 10. An epithelium 32 covers the lower portions of the enamel 15 above the CEJ 20 such that the cementum 18 is not exposed in a healthy tooth 10 as shown. Connective tissue 34 of the gingiva tissue 24 is adjacent to the epithelium 32 (this portion is referred to as the junctional epithelium 33) and includes a bed of capillaries, hereinafter referred to as a capillary bed 36, and alveolar fibers 38. An alveolar bone 40 has an alveolar crest 42 that extends nearly to the CEJ 20 to form a deep socket (not shown) for the tooth 10. Biofilm 44 generally forms to coat the outer surface of the enamel 15. FIGS. 2A and 2B illustrates a close-up of an environment 46 about the CEJ 20 including the location of the junctional epithelium 33 between the enamel 15 and the connective tissue 34 that includes the capillary bed 36.

In contrast, FIG. 1B illustrates the same tooth 10 of FIG. 1 but where periodontal disease has resulted in the loss of the alveolar bone 20B and associated gingiva 22B. The alveolar bone 40B has decreased with a reduction in the height of the alveolar crest 40B. As such, there is considerably less connective tissue 34B of the gingiva tissue 24B that is adjacent to the epithelium 32B and also a smaller capillary bed 36B and far fewer alveolar fibers 38B creating a pocket 37. The alveolar crest 42B has reduced and exposes the CEJ 20 and can in some cases expose the socket (not shown) for the tooth 10. As shown, where such losses occur, the cementoenamel junction (CEJ) 20B can widen forming the pocket 37 and exposing the lower end of the enamel 15 and a portion of the cementum 18, shown as exposed tooth portion 39. As shown, the gum 12B has less gingiva 22B including less gingival tissue 24B and increased or widened gingival sulcus 30B at the intersection of the gum 12 with the tooth 10. With the widened gingival sulcus 30B, the epithelium 32B that covers the lower portions of the enamel 15 above the CEJ 20 such that the cementum 18 can become exposed as shown.

FIGS. 2A and 2B illustrates a close-up of an environment 46 about the CEJ 20 including the location of the junctional epithelium 33 between the enamel 15 and the connective tissue 34 that includes the capillary bed 36.

FIGS. 3A and 3B and 4A and 4B illustrate environment 46 in additional detail. Where the epithelium 32 exists, as shown at “A” in FIG. 3B, there is only 1 or 2 cells separating the interface with the outer surfaces of the tooth 10 that can be exposed as exposed portion 39 due to the pocket 37 when the gingival sulcus 30 widens during infection. When the bacteria proliferate in this region, the tissue is modified so there is no epithelium 32 present as shown in FIGS. 4A and 4B at “B”. The exposure of the underlying connective tissue 34 and capillary system 36 allows for direct contact or at least very close proximity of any pathogens present in the gingival sulcus 30 or, a periodontal pocket (not shown) associated therewith. As such, there is direct access to the host circulatory system by such bacteria and thus systemic involvement is possible.

As shown in FIG. 1B, a diseased tooth 10 such as with one having an inflammation from periodontal disease, the junctional epithelium 32 moves apically (or toward the apex of the root), exposes the cementum 18 as part of portion 39, and enlarges the sulcus 30 thereby creating the pocket 37. Facultative anaerobes modify this environment 46 from one with minimal oxygen to an anaerobic environment conducive to development of virulent anaerobic periopathogens. An ulcer 48, such as shown under magnification in FIGS. 4A and 4B can form in the periodontal pocket 37. As can be seen in this illustration, the ulcer 48 of the periodontal pocket 37 can be directly adjacent to, and/or in contact with the capillary bed 36 of the connective tissue 34. As the inflammation spreads, the alveolar bone 40 is destroyed. This increases the mobility of the tooth 10 and can lead to loss of the tooth 10. Additionally, this enlarging reservoir (enlarged sulcus 30 and pocket) serves as a source of bacteria, bacterial products, and host inflammatory responses that can become systemically involved via the close proximity to the host bloodstream. The loss of alveolar bone 40 can be reversed with the application of the current method and system.

The method includes preparing a periodontal medicament delivery tray for the patient wherein the delivery tray has one or more application regions and can have one or more tooth indentations that aid in the receiving of medicaments and the delivery of the medicaments to desired specific and differentiated treatment regions so as to target the delivery of selected medicaments for treatment of the specific bacteria within the biofilm as determined for each treatment region. The periodontal medicament delivery tray therefore will be prepared so as to include delivery regions that are specifically matched to the desired treatment regions. As described herein, a periodontal medicament delivery tray may be one that is capable of delivering the selected medicament to each treatment region, such as periodontal pockets 37 associated with each tooth, and maintaining the medications in the treatment region for a sufficient period of time so as to effectively treat the bacteria within the biofilm.

One example, of such is the Perio Tray™ as described in U.S. Pat. No. 6,966,773, as issued to the inventor hereof. As described herein, such periodontal delivery trays, or variations thereof, can be worn by the patient for the purpose of controlling the oral periopathogens that are associated with specific systemic disease factors and conditions. Referring to FIGS. 5, 6 and 7, a form-fitted flexible periodontal medicament delivery tray 50, is adapted for applying the hydrogen peroxide and/or one or more of the medicaments 52 described in the various methods in accordance with various embodiments. More specifically, tray 50 is of a suitable soft plastic elastomeric or other suitable material which is molded in place to the patient's teeth so as to form a dental arch recess 54 which conforms closely to a patient's teeth and which firmly and closely fits in place on the patient's teeth. Tray 50 is shown to be a full arch tray, but those skilled in the art will recognize that a partial arch tray or a dual arch tray may be used, if desired.

The method and system herein can be performed in any suitable manner that may be equivalent to the delivery tray. When the delivery tray is used, a tray is adapted for directly applying the hydrogen peroxide and/or a medicament to the gums about the teeth, the gingival tissue, and into the periodontal pocket. For example, the medicament can be filled or at least partially filled into a cavity of periodontal medicament delivery tray. The tray can then be placed over the teeth and/or applied to the gums and about the associated teeth for application of the medicament to the area to be treated. Additionally, as shown in FIG. 5, in a region where there is only a single tooth and/or treatment region, only one of the upper or lower teeth and associated gingival tissue will be treated. In the alternative, where both the upper and lower teeth include one or more treatment regions, as shown in FIG. 6, both the upper and lower teeth and associated gingival tissue can be treated simultaneously.

Referring again to the drawings, FIGS. 8A and 8B are a close-up photographic image (FIG. 8A) and a graphical representation thereof (FIG. 8B), respectively, and FIGS. 10A and 10B are a side image radiograph (FIG. 10A) and graphical representation thereof (FIG. 10B) of a patient taken in 1976 when the patient had the bone level for a 55 year old that shows the patient is beginning to experience bone loss and tissue recession about the tooth 10 due to age. As shown in 1976, there is no laminar dura and the surface of the bone 40, especially around the maxillary posterior molars with beginning erosion. One would expect a further degeneration of the bone 40 support over time with a resultant decrease in total bone 40. As shown, the teeth 10 appear elongated with considerably recessed gingival tissue and noticeable periodontal pockets 37 with exposed portions 39 of the teeth.

Through application of the present methods including the use of topically applied doxycycline from 1976 to 2012, FIGS. 9A and 9B and FIGS. 11A and 11B illustrate the same patient images taken in the 2012 radiograph image that reflect not only a decrease in the loss of the bone 40 but an increase or regrowth of the alveolar bone 40 even though the patient has aged significantly since 1976 (e.g., 37 years later). As shown in FIGS. 8A and 8B and 10A and 10B, the alveolar bone 40 had begun to recede before treatments. However as shown in corresponding FIGS. 9A and 9B and 11A and 11B, respectively, after receiving regular treatment of hydrogen peroxide and an antibiotic, the alveolar bone 40 has not lost mass but to the contrary has regrown and increased as shown relative to the crown of the teeth 10. The photographic imaged of FIGS. 9A and 11A reflect observable increases in the bone 10 after the bone 10 had been treated with the doxycycline and such bone growth is observable as the radio-opaque layer around the teeth 10. The application of the present method reverses the concept of bone loss over time as osteoclasts are inhibited and osteoblasts are allowed to continue bone ossification. The more bone support that is regenerated is accompanied with an increased tissue development which reduces the amount of tooth 10 that is exposed as portion 39, and reduced the periodontal pockets 37, thus decreasing the gum recession that is sometimes observed as patients lose bone over time.

Similarly, FIGS. 12A and 12B are a photographic image radiograph (FIG. 12A) and a graphical representation thereof (FIG. 12B) of a patient taken in 2004 showing the effects of alveolar bone loss and its effect on the loss of gingival tissue due to periodontal disease before beginning the herein disclosed treatments. FIGS. 14A and 14B are a right side photographic image (FIG. 14A) and graphical representation thereof (FIG. 14B) of the same patient corresponding to FIGS. 12A and 12B also taken in 2004 and FIGS. 16A and 16B are a left side photographic image (FIG. 16A) and graphical representation thereof (FIG. 16B) of the patient corresponding to FIGS. 12A and 12B also taken in 2004.

The initial photograph demonstrates significant tissue recession and increases of exposed tooth portions 39, and increases in the periodontal pockets 37, especially evident for the maxillary and mandibular cuspid teeth. The fact teeth appear longer as we age is supported in the literature. This is explained as a greater action of osteoclasts as compared to the cellular activity of osteoblasts with a resultant decrease in alveolar bone around the patient's teeth with an accompanying loss of gingival tissue appearing as receding gum tissue and the exposure of a greater root surface (portions 39) making the teeth appear longer. Gum recession is very apparent before the patient began the presently described methods.

FIGS. 13A and 13B are a front photographic image (FIG. 13A) and graphical representation thereof (FIG. 13B) of the same patient of FIGS. 12A and 12B taken in 2012 after having received the herein described treatments and showing the positive effects of growth of the alveolar bone and growth of gingival tissue thereon demonstrating the effects of the current method of treatment using hydrogen peroxide and doxycycline as disclosed by way of one embodiment.

FIGS. 15A and 15B are a right side photographic image (FIG. 15A) and graphical representation thereof (FIG. 15B) of the patient corresponding to FIGS. 14A and 14B also taken 2012 (the same as FIGS. 13A and 13B) after receiving treatment for 8 years.

FIGS. 17A and 17B are a left side photographic image (FIG. 17A) and graphical representation thereof (FIG. 17B) of the patient corresponding to FIGS. 16A and 16B also taken 2012 (the same as FIGS. 13A and 13B) after receiving treatment for 8 years. FIGS. 18A and 18B are a lateral perspective radiograph image (FIG. 18A) and graphical representation thereof (FIG. 18B) of the patient of FIGS. 12A and 12B also taken in 2004.

After the photographs of FIGS. 12A, 14A, 16A, and 18A were taken in 2004, the patient began a treatment as described above that included regular direct/topical medication delivery to the a to the gingival sulcus and/or periodontal pocket 37 in 2004 delivering doxycycline subgingival and interproximal with the medicament delivery tray.

After treatment from 2004, the images of FIGS. 13A, 15A, 17A, and 18A (reflected in the corresponding graphical representations of FIGS. 13B, 15B, 17B, and 18B respectively, showing the clear positive effects of such treatment on the growth of the alveolar bone 40. This growth is clear from a comparison of the pre-treatment images FIG. 12A as compared to FIG. 13A, FIG. 14A as compared to FIG. 15A, FIG. 16A as compared to FIG. 17A, and FIG. 18A as compared to FIG. 19A, the pretreatment images with reduced bone state 40B and reduced gingival tissue 24B, increased exposed tooth portions 39 and increased periodontal pocket 37 depth as compared to the greater presence of bone 40 and increased gingival tissue 24. These clearly reflect positive effects of the current method of treatment using hydrogen peroxide and doxycycline as disclosed by way of one embodiment. This patient has been treated using hydrogen peroxide (Perio Gel) and doxycycline 50 gm/5 ml. FIGS. 12A, 14A, 16A, and 18A illustrate for this patient significant gingival tissue recession 24B, especially evident for the maxillary and mandibular cuspid teeth. The fact teeth appear longer as we age is supported in the literature. This is explained as a greater action of osteoclasts as compared to the cellular activity of osteoblasts with a resultant decrease in alveolar bone 40B around the patient's teeth 10 with an accompanying loss of gingival tissue 24B appearing as receding gum tissue 24B and the exposure of a greater root surface making the teeth 10 appear longer. This process has been reversed by the treatment of hydrogen peroxide and doxycycline as reflected in FIGS. 13A, 15A, 17A, and 19A.

As can be seen, gum recession is very apparent before the patient began using a direct medication delivery to the gingival sulcus or periodontal pocket 37 in 2004. The treatment of delivering doxycycline subgingival and interproximal with the Perio medicament delivery tray topically and directly to the tooth and surround gums has had a very positive impact on the periodontal disease. The present method of creating an oxygen rich environment using hydrogen peroxide and then the application of doxycycline clearly resulted in the inhibition of the function of the osteoclasts, thus decreasing the loss of alveolar bone. Osteoclasts precursor cells have been completely stopped from making any new osteoclasts. The net result of these actions is the stoppage of bone loss. The osteoblasts have migrated to the vacated osteoclastic position and have initiated new bone formation that is reflected in growth of gum tissue and less exposed portions 39. All of these actions have been shown to decrease the patient's bone loss and augment bone regeneration. The result is the visual appearance of less root surface exposure (less exposed portions 39) and/or decreased gingival recession as it is obvious the maxillary and mandibular teeth no longer appear as long as the bone and tissue have been restored to a more normal level.

After application of antioxidant Vibramycin over the treatment period along with a broad spectrum antibiotic such as doxycycline topically using the medicament delivery tray, the regions of root exposure appear have decreased due to growth of new alveolar bone 40 and the new alveolar tissue 24 that has also grown on the new alveolar bone 40 that makes the alveolar tissue 24 appear to be less recessed and the teeth 10 appear to have less root exposure and are therefore shorter in appearance. FIGS. 15A and 15B show the same patient that started the current method after multiple treatments with conventional periodontic surgical therapy. The initial appearance of the alveolar tissues 24 demonstrates the amount of bone loss that occur when infected tissues are removed and damaged bone is surgically removed.

From these lengthy clinic tests, doxycycline has been shown herein to inhibit the attachment of osteoclasts to bone by binding to internal and external calcium ions. Existing osteoclasts detach from bone, thus stopping bone removal or loss. Osteoclastic precursor cells do not make new osteoclasts as doxycycline completely abrogates the formation of any new osteoclasts, thus eliminating the formation of new osteoclasts. Both of these circumstances limit the loss of bone. Doxycycline has been shown to maintain or increase the cellular activity of osteoblasts, resulting in an increased bone regenerative potential. It has further been shown that topical application of the doxycycline to the periodontal pocket and to the gingival tissue about the teeth following treatment of the same with hydrogen peroxide has resulted in considerable benefit in treatment of the periodontal disease, but also in the growth of the alveolar bone and then growth in new gingival tissue on the new bone growth.

As described herein, an increase in new bone makes it possible for the growth of gingival tissue covering the bone with a resultant decrease in gingival recession. As a result, esthetically the teeth appear to regain the lost gum tissue and the teeth appear shorter the longer the patient continued to manage bone loss and augment bone regeneration along with the regeneration of gum tissue to cover the new bone growth.

Heretofore, the normal expectation has been for humans as they age to experience an increased osteoclastic activity as compared with osteoblastic activity and this would result in more bone loss than bone regeneration. However, with the process and system as described herein, this process can be reversed by using a locally delivered subclinical dose of doxycycline to the gingival sulcus or periodontal pocket (Perio Protect Method) with a direct medication delivery system (Perio Protect Method). The custom formed medical delivery device (Perio Tray) delivers doctor selected medication in accordance to the scope and magnitude of the patient's conditions.

Results have demonstrated an increased development of bone and a decrease in the amount of exposed root surface. This reverses the concept of a patient's tooth becoming longer as a person ages as the clinical evidence presented shows new bone formed and new attached gingiva when patient utilize the subgingival delivery system.

Not all areas where bone loss has occurred have been able to regenerate lost bone. One reason is the development of granulomatous tissue which can occur when oral periopathogens invade the human epithelial cells. Treatments external to these cells will not control the pathogens inside the epithelial cells and this infected tissue may need to be removed before bone regeneration may be possible.

When describing elements or features and/or embodiments thereof, the articles “a”, “an”, “the”, and “said” are intended to mean that there are one or more of the elements or features. The terms “comprising”, “including”, and “having” are intended to be inclusive and mean that there may be additional elements or features beyond those specifically described.

Those skilled in the art will recognize that various changes can be made to the exemplary embodiments and implementations described above without departing from the scope of the disclosure. Accordingly, all matter contained in the above description or shown in the accompanying drawings should be interpreted as illustrative and not in a limiting sense.

It is further to be understood that the processes or steps described herein are not to be construed as necessarily requiring their performance in the particular order discussed or illustrated. It is also to be understood that additional or alternative processes or steps may be employed.

What is claimed is:

1. A method of initiating alveolar bone regrowth about a tooth of a patient suffering from bone loss due to periodontal disease, the method comprising:
topically administering to a periodontal pocket about the tooth of the patient a hydrogen peroxide solution; and
topically administering to the periodontal pocket about the tooth a medicament that is capable of managing or altering the osteoclastic and osteoblastic activities.

2. The method of claim 1 wherein the steps of topically administering the hydrogen peroxide solution and topically administering the medicament are each performed through the use of a periodontal medicament delivery treatment tray having a cavity for receiving the tooth a portion of the gum tissue surrounding the tooth including the periodontal pocket and said administering steps include placement of the hydrogen peroxide solution and the medicament in the cavity and thereafter placement of the periodontal medicament delivery treatment tray about the tooth and surrounding gum tissue.

3. The method of claim 2 wherein the placing of the hydrogen peroxide solution in the cavity of the tray and the topically administering of the hydrogen peroxide to the periodontal pocket is performed separately from the placing of the medicament in the cavity of the tray and the topically administering of the antibiotic to the periodontal pocket.

4. The method of claim 2, further comprising preparing the periodontal medicament delivery tray for the patient, the cavity of the tray having one or more application regions including one or more tooth indentations including one for the tooth.

5. The method of claim 4 wherein the tray is prepared for creating a positive pressure within the cavity when the tray is placed about the tooth and surrounding gum tissue.

6. The method of claim 1 wherein the topically administering of the hydrogen peroxide to the periodontal pocket is performed separately from the topically administering of the medicament to the periodontal pocket.

7. The method of claim 1 wherein the medicament is an antibiotic or antioxidant.

8. The method of claim 6 wherein the antibiotic or antioxidant is doxycycline.

9. The method of claim 7 wherein the doxycycline has a concentration ratio of 50 mg/5 ml.

10. The method of claim 1 wherein the medicament can be a cardiovascular medicament or another medicament with similar osteogenic control.

11. The method of claim 1 wherein the hydrogen peroxide solution is a solution of about 1.7% hydrogen peroxide.

12. The method of claim 1 wherein the method includes repeating the two steps of topically administering the hydrogen peroxide and the medicament over an extended period of time, the method further including determining that bone loss has been stopped and that the alveolar bone about the tooth has grown.

13. The method of claim 1 wherein the method includes the two steps of topically administering of the hydrogen peroxide solution and the medicament over a series of treatments inhibit the function and/or attachment of osteoclasts to the alveolar bone about the tooth and promote the cellular production of osteoblasts.

14. The method of claim 1 wherein the step of placing and applying the hydrogen peroxide solution into the periodontal pocket is for a period of time of at least about 15 minutes.

15. The method of claim 14 wherein the two steps of topically administering the hydrogen peroxide and the medicament are each repeated about 3 or 4 times per day.

16. The method of claim 1 wherein the two steps of topically administering the hydrogen peroxide and the medicament are repeated over a period of time, further comprising observing the regrowth of the alveolar bone and continuing the two steps of topically administering the hydrogen peroxide and the medicament until a desired amount of bone regrowth is observed.

17. A method of initiating bone regrowth in a patient suffering from alveolar bone loss and recessed gums about one or more teeth, comprising:
identifying a treatment region for regrowth of the alveolar bone associated the one or more teeth that includes a periodontal pocket therein;
preparing a periodontal medicament delivery tray for the patient, the delivery tray having a cavity defining one or more application regions with each application region about the one or more of the teeth in the identified treatment region;
placing a hydrogen peroxide solution in the application region of the tray cavity;
applying the hydrogen peroxide solution placed in the tray cavity to the periodontal pocket in the treatment region;
placing a medicament in the application region of the tray cavity; and
applying the medicament placed in the tray cavity to the periodontal pocket in the treatment region.

18. The method of claim 17 wherein the medicament is placed in the tray cavity with the hydrogen peroxide and wherein each step of applying can be the simultaneous applying or individually applying.

19. The method of claim 17 wherein placing and applying the medicament is a separate step from the placing and applying the hydrogen peroxide.

20. The method of claim 17 wherein the preparing of the tray is preparing the tray for creating a positive pressure within the cavity when the tray is placed about the tooth and surrounding gum tissue.

21. The method of claim 17 wherein the medicament is an antibiotic or antioxidant.

22. The method of claim 21 wherein the antibiotic or antioxidant is a doxycycline.

23. The method of claim 22 wherein the doxycycline has a concentration ratio of 50 mg/5 ml.

24. The method of claim 17 wherein the medicament is a cardiovascular medicament or similar osteogenic controlling medication.

25. The method of claim 17 wherein the hydrogen peroxide solution is a solution of about 1.7% hydrogen peroxide.

26. The method of claim 17 wherein the method includes repeating the two steps of placing and applying the hydrogen peroxide and the medicament are repeated over an extended period of time, the method further including determining that bone loss has been stopped and that the alveolar bone about the tooth has grown.

27. The method of claim 17 wherein the method includes the two steps of placing and applying the hydrogen peroxide and the medicament over a series of treatments to inhibit the function and/or attachment of osteoclasts to the alveolar bone about the tooth and promote the cellular production of osteoblasts.

28. The method of claim 17 wherein identifying a treatment region includes a technique selected from the group consisting of a DNA analysis and a PCR analysis.

29. The method of claim 17 wherein the step of applying the hydrogen peroxide solution into the periodontal pocket is for a period of time of at least about 15 minutes.

30. The method of claim 17 wherein the two steps of placing and applying the hydrogen peroxide and the medicament are repeated about 3 or 4 times per day.

31. The method of claim 17 wherein the two steps of placing and applying the hydrogen peroxide and the medicament are repeated over a period of time, further comprising observing the regrowth of the alveolar bone and continuing the placing and applying steps until a desired amount of bone regrowth is observed.
